(19) United States
(12) Patent Application Publication
(10) Pub. No.: US 2022/0319671 A1
(43) Pub. Date: Oct. 6, 2022

(54) SYSTEM, METHOD, AND APPARATUS FOR ASSISTING IN PERFORMING THERAPY USING THERAPEUTIC APPLICATION

(71) Applicant: CureApp, Inc., Tokyo (JP)

(72) Inventor: Masaki Aijima, Setagaya-ku, Tokyo (JP)

(73) Assignee: CureApp, Inc., Tokyo (JP)

(21) Appl. No.: 17/609,068

(22) PCT Filed: May 27, 2021
(86) PCT No.: PCT/JP2021/020179

§ 371 Date: Nov. 5, 2021

(30) Foreign Application Priority Data
Jun. 19, 2020 (JP) 2020-106223

(51) Int. Cl. G16H 20/70; G16H 10/20

(52) U.S. Cl. CPC G16H 20/70; G16H 10/20

(57) ABSTRACT

A system is used for assisting in performing therapy using a therapeutic application based on a life event having occurred to a user. The system includes: a health-related information storage unit that stores health-related information of a user; and an impact level storage unit that stores an impact level indicating a magnitude of an impact caused to the user by each of a plurality of life events. The system is configured to: acquire life event information indicating the life event having occurred to the user; acquire an impact level of the life event having occurred to the user, from the impact level storage unit based on the life event information; determine, based on the impact level, a maximum executable load estimated to be executable by the user; and determine therapy using a therapeutic application and to be proposed to the user performing, based on the maximum executable load.

### TECHNICAL FIELD

The present invention relates to a system, a method, a program and an apparatus for assisting in performing therapy using a therapeutic application.

### BACKGROUND ART

In recent years, a therapeutic system has been developed, and the therapeutic system operates by using a user terminal and is used for improvement of behavior related to health of a user, treatment of a disease, health management, and the like (Patent Document 1). In Patent Document 1, a system that sequentially provides an individual on a daily basis with a behavior modification message for improving poor behavior related to health, based on data collected from the individual is disclosed.

### CITATION LIST

### Patent Literature

Patent Document 1:JP 2001-92876 A

### SUMMARY OF INVENTION

### Technical Problem

A magnitude of a load of therapy differs depending on the therapy, but a known system only performs therapy selected merely based on a disease, without consideration of a magnitude of a therapeutic load of therapy executable under a condition of a user. A system that determines a magnitude of a therapeutic load of therapy executable under a condition of a user has not been realized. Thus, in a case where a magnitude of a therapeutic load is inappropriate for a current condition of a user, the user often discontinues treatment.

### Solution To Problem

The present invention has been made in view of the problems described above, and has characteristics such as the following. That is, a system in an aspect of the present invention is a system for assisting in performing therapy using a therapeutic application, based on a life event having occurred to a user, and the system includes a health-related information storage unit configured to store health-related information of a user, and an impact level storage unit configured to store an impact level indicating a magnitude of an impact caused to the user by each of a plurality of life events, and the system is configured to acquire life event information indicating the life event having occurred to the user, acquire an impact level of the life event having occurred to the user, from the impact level storage unit based on the life event information, determine, based on the impact level, a maximum executable load estimated to be executable by the user, and determine therapy using a therapeutic application and to be proposed to the user performing, based on the maximum executable load, the health-related information of the user stored in the health-related information storage unit, and a therapeutic load of therapy using a therapeutic application, or provide a system of a therapeutic application used by the user and determined based on the health-related information of the user stored in the health-related information storage unit with the maximum executable load to be used for determining therapy performed by using the therapeutic application.

Additionally, the system may further be configured to present, in response to the acquisition of the life event information, cognitive inquiry information for acquiring cognition of the user with respect to the life event, and acquire response information of the user with respect to the cognitive inquiry information, and determining a maximum executable load may include correcting the impact level based on the response information of the user acquired, and determining a maximum executable load based on the impact level corrected.

The cognitive inquiry information can include cognitive intervention information for correcting the cognition of the user.

The determining a maximum executable load may include determining a motive value of the user based on the impact level, and determining a maximum executable load based on a skill value indicating skill of the user and the motive value of the user, and the determining therapy using a therapeutic application may include determining therapy using a therapeutic application and including a therapeutic load smaller than the maximum executable load determined.

The determining a maximum executable load may include determining a maximum executable load based on Equation (1) described below, and a behavior modification threshold may be a value indicating a boundary between occurrence and nonoccurrence of behavior modification, and may be a predefined constant.

[Equation 1]

Maximum executable load=skill value−behavior modification threshold/motive value (1)

Information proposing the therapy using the determined therapeutic application may be presented to the user.

A method in an aspect of the present invention is a method executable by a system for assisting in performing therapy using a therapeutic application, based on a life event having occurred to a user, and the system includes a health-related information storage unit configured to store health-related information of a user, and an impact level storage unit configured to store an impact level indicating a magnitude of an impact caused to the user by each of a plurality of life events, and the system includes acquiring life event information indicating the life event having occurred to the user, acquiring an impact level of the life event having occurred to the user, from the impact level storage unit based on the life event information, determining, based on the impact level, a maximum executable load estimated to be executable by the user, and determining therapy using a therapeutic application and to be proposed to the user performing, based on the maximum executable load, the health-related information of the user stored in the health-related information storage unit, and a therapeutic load of therapy using a therapeutic application, or providing a system of a therapeutic application used by the user and determined based on the health-related information of the user stored in the health-related information storage unit with the maximum executable load to be used for determining therapy performed by using the therapeutic application.

A program in an aspect of the present invention can be a program for causing a computer to execute the method described above.

An apparatus in an aspect of the present invention is an apparatus for assisting in performing therapy using a therapeutic application, based on a life event having occurred to a user, and the system includes a health-related information storage unit configured to store health-related information of a user, and an impact level storage unit configured to store an impact level indicating a magnitude of an impact caused to the user by each of a plurality of life events, and the apparatus is configured to acquire life event information indicating the life event having occurred to the user, acquire an impact level of the life event having occurred to the user, from the impact level storage unit based on the life event information, determine, based on the impact level, a maximum executable load estimated to be executable by the user, and determine therapy using a therapeutic application and to be proposed to the user performing, based on the maximum executable load, the health-related information of the user stored in the health-related information storage unit, and a therapeutic load of therapy using a therapeutic application, or provide a system of a therapeutic application used by the user and determined based on the health-related information of the user stored in the health-related information storage unit with the maximum executable load to be used for determining therapy performed by using the therapeutic application.

### Advantageous Effects of Invention

According to the present invention, a user can be assisted in behaving or performing behavior modification according to therapy appropriate for an executable load of the user by proposing therapy or the like using a therapeutic application in consideration of a maximum executable load estimated based on a life event having occurred to the user or by providing a system of a therapeutic application used by the user with the maximum executable load to be used for determining therapy performed by using the therapeutic application.

### BRIEF DESCRIPTION OF DRAWINGS

FIG. 1 is a configuration diagram of a system according to an embodiment of the present invention.

FIG. 2 is a hardware configuration diagram of a user terminal, a user information management server, and a therapy performance assistance server according to an embodiment of the present invention.

FIG. 3 is a functional block diagram of a user terminal, a user information management server, and a therapy performance assistance server according to an embodiment of the present invention.

FIG. 4 is a conceptual diagram illustrating a relationship between a motive and execution skill for successful behavior and behavior modification.

FIG. 5 is a flowchart according to an embodiment of the present invention.

### DESCRIPTION OF EMBODIMENTS

### First Embodiment

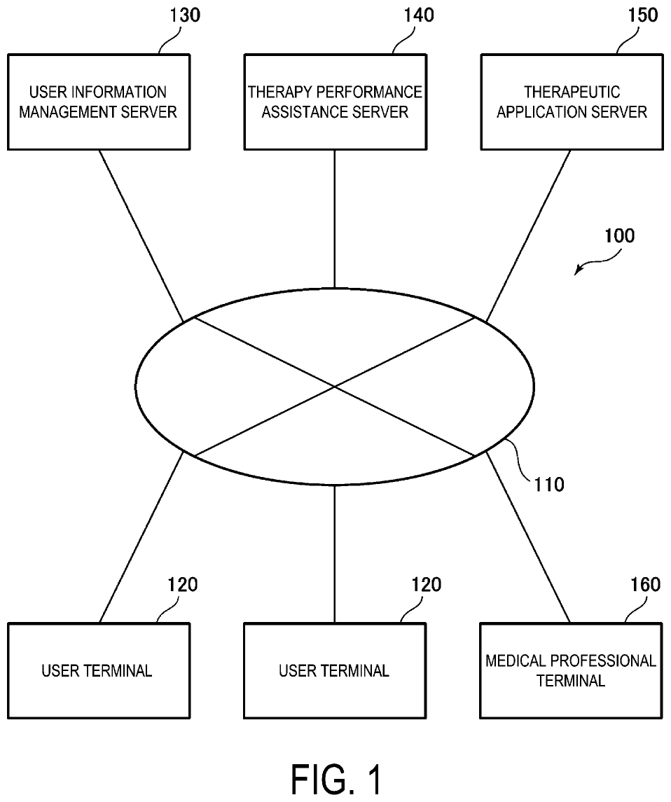

FIG. 1 illustrates an example of a system configuration diagram of the present invention. A system 100 can be used for assisting in performing therapy using a therapeutic application, based on a life event having occurred to a user, and the system 100 includes a network 110, a user terminal 120, a user information management server 130, a therapy performance assistance server 140, a therapeutic application server 150, and a medical professional terminal 160.

The user terminal 120 is a terminal of a user operated by a user of the present system, and the user information management server 130 is a server including a function to manage information of the user, and the therapy performance assistance server 140 is a server for presenting therapy suitable for the user and assisting the user in performing therapy using a therapeutic application. The therapeutic application server 150 is a server for managing a therapeutic application installed in the user terminal 120. The medical professional terminal 160 is a terminal used by a medical professional such as an attending physician of the user.

In the present embodiment, the therapeutic application means an application that makes the user execute preferable behavior related to health of the user, or that makes the user improve behavior by behavior modification, or that is used for treatment of a disease or health management, but any application may be used as long as the application is used to improve a health condition of the user. The therapeutic application is not limited to an application provided by a medical professional or used under management of a medical professional.

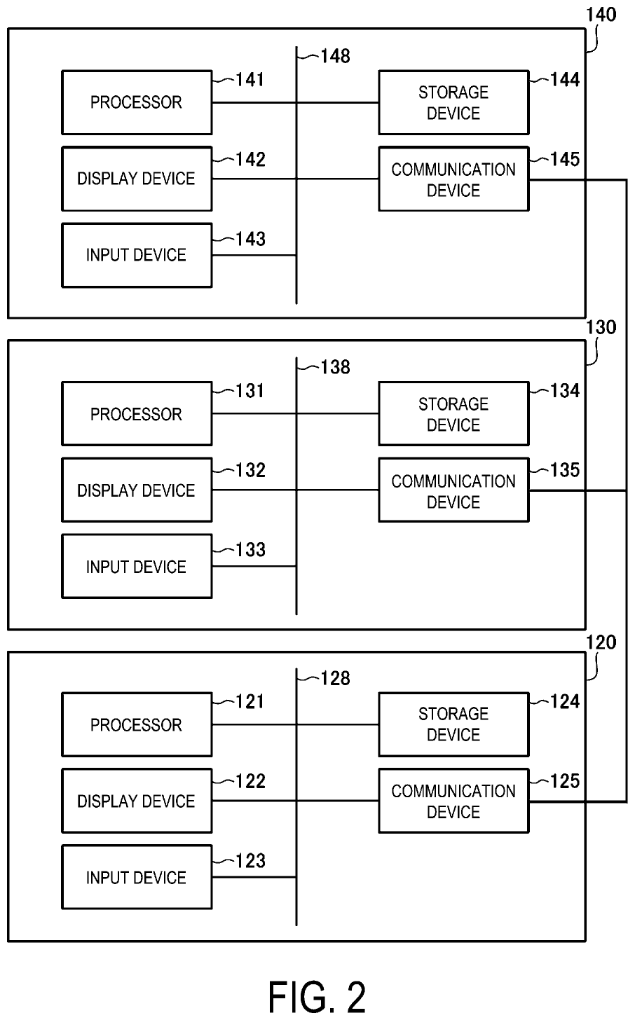

FIG. 2 is a block diagram illustrating hardware configurations of the user terminal 120, the user information management server 130, and the therapy performance assistance server 140 according to an embodiment of the present invention. The user terminal 120 includes a processor 121, a display device 122, an input device 123, a storage device 124, and a communication device 125. Each of these component devices is connected by a bus 128. Note that it is assumed that an interface is interposed between the bus 128 and each of the component devices as necessary. In the present embodiment, the user terminal 120 is a smartphone. However, the user terminal 120 can be an information terminal such as a personal computer and a tablet computer, as long as the user terminal 120 includes the configuration describe above.

The user information management server 130 and the therapy performance assistance server 140 also similarly include processors 131, 141, display devices 132, 142, input devices 133, 143, storage devices 134, 144, and communication devices 135, 145, respectively. Each of these component devices is connected by a bus 138, 148. Note that it is assumed that an interface is interposed between the bus 138, 148 and each of the component devices as necessary. In the present embodiment, the user information management server 130 and the therapy performance assistance server 140 are realized by a computer.

The processor 121, 131, 141 controls all operations of the user terminal 120, the user information management server 130, and the therapy performance assistance server 140, and is, for example, a CPU. The processor 121, 131, 141 executes various types of processing by reading and executing a program and data stored in the storage device 124, 134, 144. In an example, the processor 121, 131, 141 includes a plurality of processors.

The display device 122, 132, 142 displays an application screen and the like to the user (patient) of the user terminal 120, and to a user (administrator) of the user information management server 130 and the therapy performance assistance server 140, according to control of the processor 121, 131, 141. The display device 122, 132, 142 is preferably a liquid crystal display, but may be a display using organic EL, a plasma display, or the like.

The input device 123, 133, 143 is a user interface that receives an input from the user with respect to the user terminal 120, the user information management server 130, and the therapy performance assistance server 140, and the input device 123, 133, 143 is, for example, a touch panel, a touch pad, a keyboard, or a mouse. In the present embodiment, the user terminal 120 is a smartphone, and thus, the user terminal 120 includes a touch panel as the input device 123, and the touch panel also functions as the display device 122, and the display device 122 and the input device 123 are integrated. The display device 122 and the input device 123 may be in a separate form in which the display device 122 and the input device 123 are disposed at separate positions. Here, the user information management server 130 and the therapy performance assistance server 140 are computers, and thus it is assumed that the user information management server 130 and the therapy performance assistance server 140 each include a keyboard and a mouse as the input device, and a liquid crystal display as the display device.

The storage device 124, 134, 144 is a storage device including a RAM that is a volatile memory and a ROM that is a non-volatile memory, and being provided in a common smartphone or computer. The storage device 124, 134, 144 can also include an external memory. For example, the storage device 124 stores a user application for performing the present embodiment, and the storage device 134, 144 stores a server application. The user application includes a user program such as a web browser application and a therapeutic application, and various types of data referred to during execution of the user program. The user program is started in response to an operation of the user with respect to the user terminal 120, and is executed on an operating system (OS) mounted in advance on the user terminal 120. The application for a server includes a function and various types of data for causing reception of an input from the user and presentation of information to the user to be appropriately executed by the user program in each user terminal 120.

In an example, the storage device 124, 134, 144 includes a main storage device and an auxiliary storage device. The main storage device is a volatile storage medium to and from which information can be written and read at high speed, and is used as a storage area and a work area when the processor 121, 131, 141 processes information. The auxiliary storage device stores various programs, and data used by the processor 121, 131, 141 during execution of each program. The auxiliary storage device is, for example, a hard disk device, but may be any non-volatile storage or nonvolatile memory as long as the non-volatile storage or the nonvolatile memory can store information, and the auxiliary storage device may be removable. The auxiliary storage device stores, for example, an operating system (OS), middleware, an application program, and various types of data that can be referred to in association with execution of these programs.

The communication device 125, 135, 145 transmits and receives data to and from another device via the network 110 (omitted in FIG. 2). For example, the communication device 125, 135, 145 performs mobile communication and wireless communication via a wireless LAN or the like, and is connected to the network 110. The user terminal 120 and the medical professional terminal 160 use the communication devices 125 and 135, respectively, and perform communication with the user information management server 130 via a network. The communication device 125, 135, 145 may perform wired communication using an Ethernet (registered trademark) cable or the like.

A hardware configuration of the therapeutic application server 150 is similar to the hardware configuration of the user terminal 120, and a hardware configuration of the medical professional terminal 160 is similar to the hardware configurations of the user information management server 130 and the therapy performance assistance server 140, and thus illustration and detailed description of the hardware configurations of the therapeutic application server 150 and the medical professional terminal 160 are omitted.

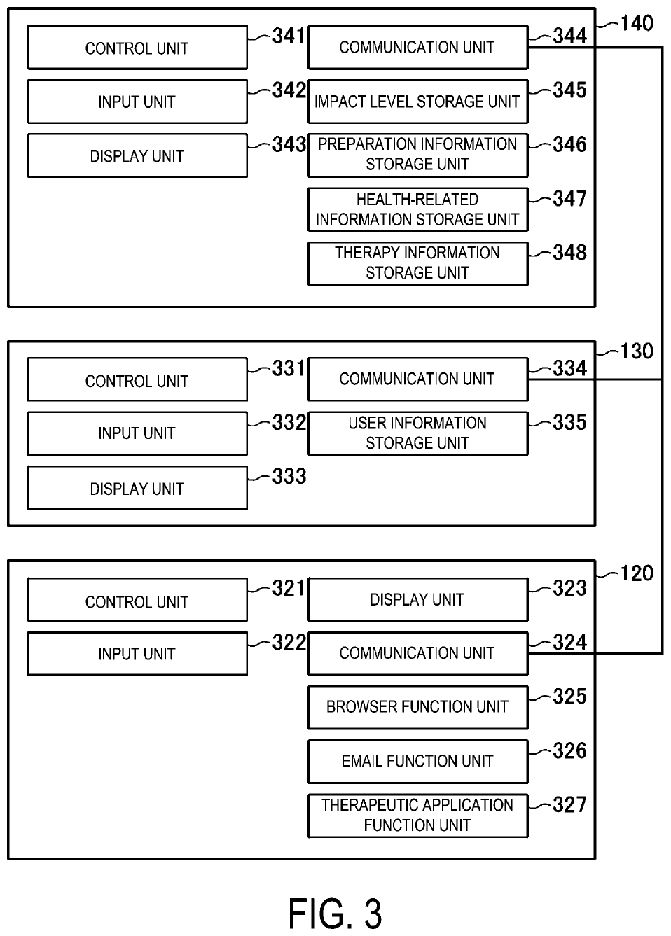

FIG. 3 illustrates an example of a functional block diagram of the user terminal 120, the user information management server 130, and the therapy performance assistance server 140 according to an embodiment of the present invention. The user terminal 120 includes a control unit 321, an input unit 322, a display unit 323, a communication unit 324, a browser function unit 325, an email function unit 326, and a therapeutic application function unit 327, and the user information management server 130 includes a control unit 331, an input unit 332, a display unit 333, a communication unit 334, and a user information storage unit 335, and the therapy performance assistance server 140 includes a control unit 341, an input unit 342, a display unit 343, a communication unit 344, an impact level storage unit 345, a preparation information storage unit 346, a health-related information storage unit 347, and a therapy information storage unit 348.

In the present embodiment, these functions are realized by causing the processor 121, 131, 141 to execute a program. For example, the program executed is a program stored in the storage device 124, 134, 144. In this way, various types of functions are realized by reading a program, and thus, a portion or all of one part (function) may be provided in another part. These functions may be realized by hardware by constituting an electronic circuit or the like for realizing a part or all of each function.

The control unit 321 of the user terminal 120 performs control processing of information processing executed in the present embodiment. The input unit 322 receives an input from the user of the user terminal 120. The display unit 323 displays an application screen for controlling a function of an application, and displays an application screen corresponding to a function of an application and a user operation. In the present embodiment, a touch panel including the input unit 322 and the display unit 323 is used, and an operation receiving unit is realized by a touch detection function. The communication unit 324 performs communication with other user terminal 120, the user information management server 130, the therapy performance assistance server 140, and the like by wired and wireless communication.

The browser function unit 325 is a function realized by a general browser application for accessing a web page of a server and the like connected via the network 110 such as the Internet. In the present embodiment, the browser function unit 325 used by the user includes functions to access the user information management server 130 and the therapy performance assistance server 140, and update user information and to present a notification and the like from a server to the user. The email function unit 326 includes a function to transmit and receive an email via the network 110. The therapeutic application function unit 327 includes a function to perform treatment by, for example, acquiring information from the user, or presenting information to the user while communicating with the therapeutic application server 150 as necessary.

The control units 331, 341 of the user information management server 130 and the therapy performance assistance server 140 perform control processing of information processing executed in the present embodiment. The input unit 332, 342 receives an input from the user of each server, and the display unit 333, 343 displays information to the user of each server. The communication unit 334, 344 performs communication with the user terminal 120, other server and the like by wired and wireless communication.

The user information storage unit 335 of the user information management server 130 stores information of the user. For example, the user information storage unit 335 stores information related to the name, age, and gender of the user, and related to a life event having occurred to the user. In the present embodiment, the user information management server 130 is a server that manages and stores personnel information of an employee in a company. The personnel information can include, in addition to the name, age, and gender of the user that is an employee, presence or absence of a spouse, presence or absence of a child, a work location, or the like. Examples of the information related to a life event having occurred to the user can include information of marriage of the user, death having bereaved of a spouse or a child, and job transfer.

The impact level storage unit 345 of the therapy performance assistance server 140 stores information indicating an impact level with respect to a life event. The impact level is a factor that affects a magnitude of a motive of the user to behave or execute behavior modification. A magnitude of the impact level is predefined with respect to each life event. In the present embodiment, the impact level storage unit 345 stores a life event-impact level table that associates a life event with a magnitude of the impact level corresponding to the life event. An example of the life event-impact level table is indicated in Table 1.

TABLE 1

| | | Standard |
|---|---|---|
| ID | Life event | impact level |
| 0 | Death of spouse | −90 |
| 1 | Divorce | −70 |
| 2 | Marital separation | −65 |
| 3 | Death of close relative | −63 |
| 4 | Serious injury | −53 |
| | and illness of user | |
| 5 | Marriage | 50 |
| 6 | Loss of | −47 |
| | employment | |
| 7 | Retirement | 45 |
| 8 | Change of | −44 |
| | family health | |
| 9 | Pregnancy | 40 |
| 10 | Death of friend | −37 |
| 11 | Job relocation and | 36 |
| | transfer (positive) | |
| 12 | Job relocation and | −36 |
| | transfer (negative) | |
| 13 | Debt and | −30 |
| | foreclosed loan | |
| 14 | Birth of child | 30 |
| 15 | Personnel reshuffle | −29 |
| 16 | Marriage of child | 29 |
| 17 | Employment and | 26 |
| | retirement of wife | |
| 18 | Education | 26 |
| | advancement | |
| | and graduation | |
| 19 | Change of | 24 |
| | personal habit | |
| 20 | Trouble with superior | −23 |
| 21 | Change of working | 20 |
| | hours and working | |
| | condition | |
| 22 | Change of place | −20 |
| | of residence | |
| 23 | Change of school | 20 |
| 24 | Debt equal to or less | −17 |
| | than million yen | |
| 25 | Change of number of | 15 |
| | family members | |

In Table 1, the ID is identification information being in correspondence with each life event, and the standard impact level indicates a magnitude of a standard impact level predefined with respect to a life event being in correspondence. A magnitude of the impact level caused to a person by a life event may differ for each person, but the magnitude of the standard impact level can be defined. Here, the standard impact level is stored in correspondence with each life event as the magnitude of the standard impact level of each life event.

In general, a life event includes a life event that is likely to be associated with positive cognition, and a life event that is likely to be associated with negative cognition. For example, while birth of a child becomes an opportunity of a positive challenge, death having bereaved of a spouse indicates a negative tendency to escape. In the present embodiment, a negative value is defined as the impact level with respect to a life event associated with negative cognition, and a positive value of the impact level is defined with respect to a life event associated with positive cognition. For example, “Death of spouse” is in correspondence with life event ID=0, and an impact level of the life event is “−90”. Death of a spouse generally means a life event that causes a significant negative impact.

The preparation information storage unit 346 of the therapy performance assistance server 140 includes preparation information for correcting the impact level before presenting a therapeutic application to the user. An example of life event-preparation information in the present embodiment is indicated in Table 2.

TABLE 2

| | | | | Impact level |
|---|---|---|---|---|
| ID | Life Event | Question | Response | correction value |
| 0 | Death of spouse | Successful | Yes | −1 |
| | | switch of | Unsure | 1 |
| | | feeling? | No | 1 |
| 1 | Divorce | What kind of | Positive | −1 |
| | | event is divorce | Unsure | 1 |
| | | for you? | Negative | 1 |
| ... | ... | ... | ... | ... |
| 14 | Birth of child | What kind of | Positive | 1 |
| | | event is birth of | Unsure | 1 |
| | | child for you? | Negative | −1 |
| ... | ... | ... | ... | ... |

In Table 2, the ID is a life event ID, and a question, a response, and an impact level correction value with respect to each response that are cognitive inquiry information are predefined with respect to the life event ID. For example, a life event of ID=0 is “Death of spouse”, and a question is “Successful switch of feeling?” and three response choices “Yes”, “Unsure”, and “No” are defined as responses, and impact level correction values “−1”, “1”, and “1” are defined with respect to the respective responses. Examples of the preparation information may include cognitive intervention information that causes the user motivation to perform therapy using a therapeutic application. The impact level correction value can be set appropriately, and, for example, an impact level correction value may be “2” with respect to the response of “No”, and the response of “No” may be distinguished from the response of “Unsure”.

The health-related information storage unit 347 of the therapy performance assistance server 140 stores health-related information related to health of the user. For example, the health-related information related to health of the user can include the gender, age, weight, contact, medication history, past medical history, current disease, treatment history, and the like of the user. The treatment history can include information indicating a therapeutic application currently used. The current disease is not required to be illness in a strict medical sense, and may include a smoking habit and the like. For example, these pieces of information may be input via the user terminal 120 by the user having consented to reception of provision of therapy performance assistance service provided by the therapy performance assistance server 140, or may be input via the medical professional terminal 160 used by a physician or the like. These pieces of information may be based on information acquired by a medical checkup in a company. As the information stored by the user information management server 130, information of the user information management server 130 may be referred to.

The therapy information storage unit 348 stores information related to therapy presented to the user. In present embodiment, the information related to therapy includes information specifying a therapeutic application, information specifying therapy executed by each therapeutic application, and information indicating a load of each type of therapy. The load of therapy indicates a level of skill and the like required to behave and execute behavior modification according to the therapy, and is predetermined based on information provided by a therapeutic application, a level of difficulty in learning a technique such as coping, and the like. An example of a therapy table indicating therapy information is indicated in Table 3.

TABLE 3

| ID | Disease | Therapeutic Application | Therapy | Load |
|---|---|---|---|---|
| 0 | Smoking habit | Application A | Therapy α | 99 |
| 1 | Smoking habit | Application B | Therapy β | 97 |
| 2 | Smoking habit | Application B | Therapy θ | 94 |
| 3 | Fatty liver | Application C | Therapy δ | 93 |
| 4 | Hypertension | Application D | Therapy ε | 99 |
| 5 | Hypertension | Application D | Therapy Φ | 98 |
| 6 | Hypertension | Application E | Therapy γ | 96 |
| 7 | Hypertension | Application F | Therapy η | 94 |

In Table 3, the ID is identification information assigned to each type of therapy, the disease indicates a disease to be treated by each type of therapy, the therapeutic application is information identifying a therapeutic application, the therapy is information indicating therapy performed by a therapeutic application, and the load means a load of therapy. For example, ID=0 means that therapy is therapy for smoking cessation treatment with respect to a smoking habit, and is therapy α using an application A, and that a load of the therapy is 99. One therapeutic application may be in correspondence with a plurality of types of therapy. The load with respect to therapy is predetermined. As contents of treatment is more comprehensive, a larger treatment load can be set, and as contents of treatment is more moderate and limited, a smaller treatment load can be set. Additionally, the identification information may include information of a therapeutic application alone, and may not include information of therapy performed by the application. It can be considered that when a therapeutic application is specified, therapy executed by the therapeutic application is specified.

The user information storage unit 335 in the user information management server 130, and the impact level storage unit 345, the preparation information storage unit 346, the health-related information storage unit 347, and the therapy information storage unit 348 in the therapy performance assistance server 140 may be realized by a storage device of each server or may be realized by a database provided separately from each server.

In the present embodiment, it is assumed that a web browser application and a therapeutic application are installed in each user terminal 120, and perform communication with the user information management server 130, the therapy performance assistance server 140, and the therapeutic application server 150, but one user may have a plurality of the user terminals 120, and separate applications may be installed in the respective user terminals 120. For example, one user terminal 120 may include the browser function unit 325 and access the user information management server 130 to perform an input, registration, browsing and the like of information, and other user terminal 120 may include the therapeutic application function unit 327 and perform therapy while performing communication with the therapeutic application server 150.

A functional block diagram of the therapeutic application server 150 and the medical professional terminal 160 is omitted, but when a therapeutic application is executed in the user terminal 120, the therapeutic application server 150 transmits and receives data periodically or as necessary, and realizes treatment using the therapeutic application in the user terminal 120, and the medical professional terminal 160 includes a function to perform an input and an output of required information appropriately in response to an operation of a medical professional.

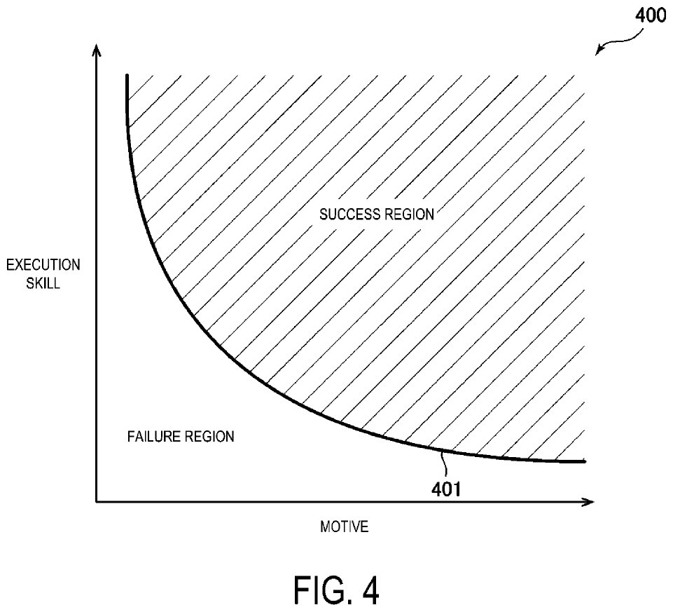

A graph 400 of FIG. 4 is a conceptual diagram illustrating a relationship between a motive and execution skill for successful behavior and behavior modification. A horizontal axis indicates a magnitude of a motive, and a vertical axis indicates a level of execution skill. As a line 401 is a boundary, a region above the line 401 is a region where behavior and behavior modification are considered to be successful, and a region below the line 401 is a region where behavior and behavior modification are considered to fail. This indicates that as a motive is larger, behavior and execution of behavior modification can be successful even when execution skill is low, and that as a motive is smaller, behavior and execution of behavior modification cannot be successful without high execution skill.

A load with respect to the user of therapy using a therapeutic application differs for each type of therapy. When behavior and behavior modification of the user required by a therapeutic application cannot be performed, effective treatment cannot be performed. When therapy using a therapeutic application is performed, the user needs to be in a condition where the user can behave and execute behavior modification by the therapy. Then, as illustrated in FIG. 4, whether the user can successfully behave and execute behavior modification depends on a magnitude of a motive and a level of execution skill. That is, a maximum value of a load under which the user can behave and execute behavior modification depends on a magnitude of a motive and a level of execution skill that the user has at that time.

A magnitude of a motive is considered to change depending on an impact level caused by a life event having occurred to the user. An impact level caused by a life event has a difference among individuals, but the impact level generally includes an impact level that is likely to be associated with positive cognition, and an impact level that is likely to be associated with negative cognition. For example, birth of a child tends to cause a large positive impact level and tends to become an opportunity of a positive challenge, and death having bereaved of a spouse tends to cause a large negative impact level and tends to indicate a negative tendency to escape.

The present invention enables execution of behavior and behavior modification by a user and performs assistance in effective therapy performance by estimating a magnitude of a motive of the user (motive value) based on an impact level caused to the user by a life event, estimating a maximum executable load of the user, and presenting therapy using a therapeutic application suitable for the user, based on the maximum executable load estimated and a load of therapy using each therapeutic application.

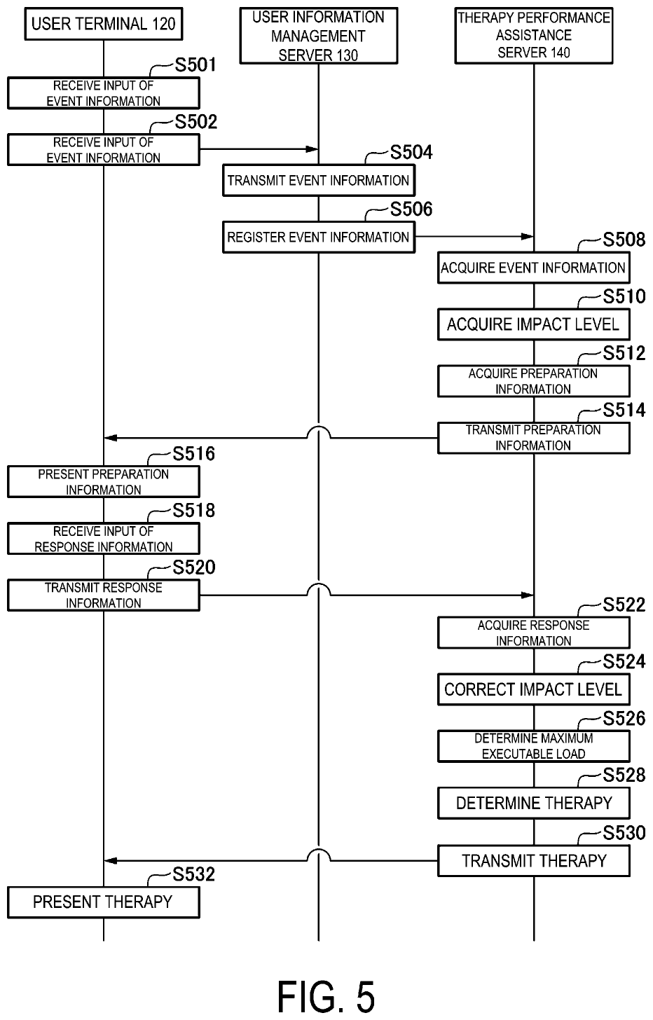

A flow of information processing in the present embodiment will be described with reference to FIG. 5. Here, the user information management server 130 is a server that manages personnel information in a company. In a personnel-related system, for payment of social insurance, various allowances, congratulatory money, and the like, information of a change in a life event such as marriage, separation, birth of a child, and job transfer is registered and updated. Information for recognizing a change in a life event of the user can be acquired accurately by using, as the user information management server 130, a server that manages personnel information.

It is assumed that to provide a therapeutic application suitable for a current condition of an employee from therapeutic applications provided by a medical institution or the like that cooperates with a company working on health maintenance and health promotion of employees, the therapy performance assistance server 140 managed by the medical institution or the like is connected to the user information management server 130 to provide personnel-related information. The therapy performance assistance server 140 and the therapeutic application server 150 may be provided within one company.

Instead of or in addition to the personnel information management server, a server of social media can also be utilized as the user information management server 130. The user often posts a change in a life event on social media to inform people around the user of the change, and thus such a server can also be utilized to acquire information about a life event on a timely basis. The therapy performance assistance server 140 may include the function of the user information management server 130, and the user may transmit a change in a life event directly to the therapy performance assistance server 140.

In the present embodiment, first, when a life event occurs to the user of the present system, the user inputs, via the user terminal 120, information specifying the life event (S501) and transmits the information input (S502), and the user information management server 130 receives the information, and stores the information in the user information storage unit 335 (S504). Event information of the user may be input by a personnel management officer in a company by using a user terminal of the personnel management officer according to a verbal or written report from the user. The user information management server 130 transmits the event information of the user to the therapy performance assistance server 140 (S506).

The therapy performance assistance server 140 acquires the event information of the user (S508), and based on the event information, the therapy performance assistance server 140 refers to the impact level storage unit 345 and acquires an impact level (S510). Further, the therapy performance assistance server 140 acquires preparation information from the preparation information storage unit 346, based on the event information acquired (S512), and transmits the preparation information to the user terminal 120 (S514). When the user receives presentation of the preparation information via the user terminal 120 (S516), the user inputs response information with respect to cognitive inquiry information provided in the preparation information (S518), and transmits the response information to the therapy performance assistance server 140 (S520).

Here, the therapy performance assistance server 140 acquires an email address of the user from contact information provided in health-related information of the user, and transmits by email link information to a web page provided by the therapy performance assistance server 140 for presenting the preparation information to the user information management server 130. It is assumed that when the user receives this email by the user terminal 120, the user accesses the web page, based on the link information to the web page, and contents of the web page including the preparation information is transmitted from the therapy performance assistance server 140, and based on the contents of the web page, the preparation information is presented to the user in the user terminal 120. The user performs, via the web page, input and transmission with respect to the inquiry information presented. The transmission and reception of the preparation information may be realized by transmission of an email and a reply with respect to the email without using the web page, or can also be performed via a chat application or other application.

When the therapy performance assistance server 140 receives the response information (S522), the therapy performance assistance server 140 performs correction with respect to the impact level acquired at S510, based on the response information (S524), and the therapy performance assistance server 140 determines a maximum executable load of the user, based on the impact level corrected (S526). The maximum executable load indicates a magnitude of a maximum therapeutic load estimated to be executable by the user. Therapy having a load equal to or more than the maximum executable load is considered to be highly likely to be unexecutable by the user, and when therapy has a load smaller than the maximum executable load, it is considered that behavior and behavior modification required by the therapy is highly likely to be executed and that a therapeutic effect is highly likely to be obtained.

The therapy performance assistance server 140 determines therapy to be proposed to the user, based on the maximum executable load determined, the health-related information of the user stored in the health-related information storage unit 347, and the load of the therapy stored in the therapy information storage unit 348 (S528), and transmits, to the user terminal 120, information about the therapy determined (S530), and the user terminal 120 presents to the user information proposing performance of the therapy, based on the information received (S532). The information about the therapy can include a therapeutic application, the therapy performed by the therapeutic application, description of an effect, an installation method of the application, and the like.

In a case where the user having received the proposal of the therapy is willing to receive the therapy, the user can install, the therapeutic application in the user terminal 120 or the like of the user, and the user can initiate the therapy. The proposal of the therapy to the user may be proposal of therapy executed by a therapeutic application by presenting information specifying the therapeutic application, or can also be proposal of therapy by specifying a portion of a plurality of types of therapy executable by the therapeutic application.

In the present embodiment, it is assumed that the impact level corrected based on the impact correction value with respect to the standard impact level is determined, and the maximum executable load is determined, but the present invention can be performed by performing estimation of the maximum executable load, based on the standard impact level without performing the impact level correction processing. Additionally, the standard impact level may be determined based on characteristics of the user such as age and gender.

According to the present embodiment, disease therapy and the like using a therapeutic application can be proposed in consideration of a maximum executable load estimated based on a life event having occurred to the user. Thus, the user can behave and execute behavior modification according to therapy appropriate for an executable load at that time, and can realize an expected therapeutic effect of the therapy. Additionally, a life event having occurred to the user can be detected, and thus a maximum executable load of the user due to influence of the life event can be estimated more accurately on a timely basis.

### EXAMPLE 1

As one example, information processing in the present embodiment will be described in more detail by exemplifying the case where a life event of birth of a child has occurred to a user A that is a smoker. It is assumed that information indicating that the user A is a smoker is recorded in advance in the therapy performance assistance server 140 as information acquired by a regular medical checkup or the like. When the user A inputs, via the user terminal 120, event information indicating birth of a child (S501) and transmits the information input (S502), the user information management server 130 receives and stores the information in the user information storage unit 335 (S504). The user information management server 130 further transmits the event information of the user A to the therapy performance assistance server 140 (S506).

When the therapy performance assistance server 140 acquires the information indicating that the life event of “Birth of child” has occurred to the user A (S508), the therapy performance assistance server 140 refers to a life event-impact level table stored in the impact level storage unit 345 and acquires “30” as an impact level of “Birth of child” (S510). Further, the therapy performance assistance server 140 refers to a life event-preparation information table stored in the preparation information storage unit 346 and acquires a question of “What kind of event is birth of a child for you?” and responses of “Yes, Unsure, No” as preparation information for “Birth of child” (S512).

The therapy performance assistance server 140 transmits by email link information to a web page for presenting presentation of the preparation information acquired, and the user accesses the web page of a link destination via the user terminal 120, and receives presentation of the question of “What kind of event is birth of a child for you?” and the response choices with respect to the question of “Positive”, “Unsure”, and “Negative” as the preparation information (S516).

When the user selects one response from the response choices and transmits the response as response information, the therapy performance assistance server 140 performs, based on the response information acquired (S522), impact level correction (S524). As described above, the impact level of “Birth of child” is “30” and is defined as a positive impact level, and thus, it is generally considered that the user has an increased maximum executable load with respect to treatment, as compared to before occurrence of the life event of “Birth of child”. However, “Birth of child” may be considered depending on persons to be a negative element such as a large burden. Then, the impact level can be corrected by acquiring cognition of the user with respect to the life event having occurred, and estimation accuracy of the maximum executable load can be increased.

In the present example, it is assumed that a corrected impact level is computed by an equation described below.

Corrected impact level=standard impact level×impact level correction value [Equation 2]

For example, in a case where the response of the user A is “Positive”, a standard impact level (30) is multiplied by an impact level correction value (+1), and a corrected impact level becomes 30, but in a case where the response of the user is “Negative”, a standard impact level (30) is multiplied by an impact level correction value (−1), and thus a corrected impact level becomes −30.

Next, based on the corrected impact level, a maximum executable load is computed by an equation described below.

Maximum executable load=skill value−behavior modification threshold/motive value [Equation 3]

Motive value=reference motive value+impact level [Equation 4]

Here, the skill value is a value indicating a level of skill of the user to execute treatment. The behavior modification threshold is a value indicating a boundary between performance and nonperformance by the user of behavior modification by treatment. The skill value and the behavior modification threshold can be determined by learning with respect to treatment, whether the user is an adult or a child, and the like. In a case where a learning level of the user is unknown, a standard value can be used. The motive value is a value indicating a magnitude of a motive of the user. Here, it is assumed that as indicated by Equation 4, the motive value is calculated by adding the impact level to a reference motive value. The reference motive value is a value indicating a magnitude of a standard motive. The reference motive value can be changed appropriately depending on a condition of the user. The standard impact level may be used as the impact level, or in a case where the impact level correction processing is executed, the corrected impact value may be used as the impact level.

In the present example, it is assumed that a maximum executable load (99.2) is acquired by setting a skill value=100, a behavior modification threshold=100, and a reference motive value=100 as standard values, and substituting a corrected impact level (30). The therapy performance assistance server 140 refers to a therapy table stored in the therapeutic information storage unit 348 and selects a therapeutic application and therapy that correspond to the disease of the user A, from therapy having a load smaller than the maximum executable load computed (99.2). Here, it is determined that therapy α using a therapeutic application A and having the highest load of loads of the therapy IDs=0 to 2 corresponding to a smoking habit as disease information provided in the health-related information of the user A is therapy to be presented to the user. Generally, therapy having a high treatment outcome tends to have a high load with respect to more people.

Here, it is assumed that therapy having a high therapeutic load is preferentially proposed, but the present embodiments are not limited to this. For example, the therapy table can separately include indicator information indicating a magnitude of a therapeutic effect, and therapy can be determined based on the indicator information. Additionally, the health-related information storage unit 347 may include disease seriousness information, and therapy with respect to a disease having high seriousness may be selected preferentially. Additionally, it is not necessary to select a single therapy alone, and a plurality of types of therapy or all types of therapy may be selected from therapy having a load equal to or less than the maximum executable load.

The therapy performance assistance server 140 transmits, to the user A, information recommending the determined therapy α using the therapeutic application A, and presents recommendation information in the user terminal 120, and thus the therapy performance assistance server 140 encourages performance of the therapy α for smoking cessation using the therapeutic application A.

The user A recognizes that birth of a child is positive, and it is considered that the user A has an increased motive for performing the smoking cessation treatment, as compared to before birth of a child. This timing is appropriately detected, and the maximum executable load of the user is estimated, and smoke cessation therapy having a load estimated to be executable is recommended, and accordingly, behavior and behavior modification can be executed according to the therapy appropriate for the executable load, while encouraging the user to initiate the smoking cessation treatment. According to the present embodiment, the user can be assisted in performing therapy.

As one modification example, a contact such as an email address of a medical professional such as an attending physician may be stored in the health-related information or the like of the therapy performance assistance server 140, and the information of the therapy using the therapeutic application and determined may be transmitted to the medical professional terminal 160 of the medical professional in addition to or instead of the user terminal 120 of the user. The physician having received proposal of the therapy can encourage the user to perform the treatment.

### EXAMPLE 2

Next, an example in a case where a user has encountered an event having a negative impact level will be described. Hereinafter, a difference from Example 1 will mainly be described. The therapy performance assistance server 140 holds, as user health-related information, information indicating that a user B has a fatty liver. The user B registers occurrence of a life event of “Death of spouse” with the user information management server 130. The therapy performance assistance server 140 receives the information of the event from the user information management server 130, and while the therapy performance assistance server 140 refers to a life event-impact level table of the impact level storage unit 345 and acquires an impact level of “−90”, of death of a spouse, the therapy performance assistance server 140 acquires, from the preparation information storage unit 346, a question of “Successful switch of feeling?” and response choices of “Yes”, “Unsure”, and “No”, in preparation information in correspondence with “Death of spouse”.

The therapy performance assistance server 140 transmits the preparation information to the user terminal 120 of the user B, and the user B selects the response choice of “No” with respect to the question, and transmits the response choice to the therapy performance assistance server 140. Based on the response received, the therapy performance assistance server 140 multiplies the impact level (−90) by an impact level correction value (1) to calculate a correction impact level (−90). The therapy performance assistance server 140 calculates a maximum executable load (90) based on the corrected impact level. Therapy having a therapeutic load equal to or less than 90 is retrieved from a therapy table, but therapy having the smallest load has a load of 93, and thus no therapy is selected and selection of therapy for the user B is not performed.

When a negative life event has occurred, it is estimated that a maximum executable load of the user is decreased. It is considered that in such a case, even when proposal of therapy having a load inappropriate for a maximum executable load is performed, the user cannot execute the therapy. According to the present example, proposal of therapy having a load inappropriate for a condition of the user can be prevented.

### Modification Example

As one modification example, as the preparation information, cognitive intervention information for correcting cognition of the user may be provided. For example, erroneous cognition of the user related to a disease of the user provided in the health-related information of the user can be detected, and information for correcting the erroneous cognition to correct cognition can be provided. Before acquisition of a response with respect to a question as the preparation information, cognition intervention can be performed. A maximum executable load can be increased by the cognition intervention, and more effective therapy can be executed.

As an example, the cognitive intervention that increases a motive for performance can be performed by clarifying a reason for willingness to receive treatment and a reason for unwillingness to receive treatment, comparing both the reasons, and causing cognition of the fact that the reason for willingness to receive treatment surpasses the reason for unwillingness to receive treatment. For example, with respect to the user requiring smoking cessation treatment, the user terminal 120 presents, based on the preparation information, information instructing the user to input a reason for willingness to perform the smoking cessation treatment and a reason for unwillingness to perform the smoking cessation treatment. The user can clearly recognize an advantage and a disadvantage of the smoking cessation treatment by the input work to the user terminal 120.

Additionally, the user may input a degree of willingness to perform the smoking cessation treatment in the range of from 0 to 100, and in a case where the degree is smaller than 100, the user may input a reason for unwillingness to perform the smoking cessation treatment. In a case where the input degree of willingness to perform the smoking cessation treatment is smaller than 100 but is not zero, the user may input a reason why willingness to perform the smoking cessation treatment is not zero. Motivation with respect to treatment can be performed by particularly clarifying a motive related to a life event.

The therapy performance assistance server 140 may transmit, to the user terminal 120, cognitive inquiry information confirming an effect of the cognitive intervention. In a case where correction of cognition by the cognitive intervention and an increased motive are detected based on response information with respect to the cognitive inquiry information, correction that increases an impact level in a positive direction can be performed.

### Second Embodiment

The present embodiment differs from the first embodiment in that, instead of the therapy performance assistance server 140 determining therapy using a therapeutic application and to be proposed to a user performing, a maximum executable load is provided to a system of a therapeutic application used by a user and determined based on health-related information of the user stored in a health-related information storage unit 347, and the maximum executable load is provided to the system to be used for determining therapy performed by using the therapeutic application. Hereinafter, a difference from the first embodiment will mainly be described.

In the present embodiment, the case where a life event occurs while a user is performing treatment by using a therapeutic application is assumed. A therapy performance assistance server 140 stores, as health-related information, information indicating that a user C has hypertension and a smoking habit and that the user C utilizes therapy φ using a therapeutic application D for hypertension. The user C registers divorce having occurred as a life event with a user information management server 130 via a user terminal 120.

When the therapy performance assistance server 140 receives event information from the user information management server 130, the therapy performance assistance server 140 refers to a life event-impact level table and acquires −70 as an impact level of divorce. The therapy performance assistance server 140 transmits preparation information to the user terminal 120 of the user C, and the user C inputs, via the user terminal 120, a response of “Positive” with respect to a question of “What kind of event is divorce for you?” and transmits the response to the user information management server 130. Based on response information received, the therapy performance assistance server 140 acquires an impact level correction value of “−1”, corrects the impact value to “+70”, and, based on the corrected impact value, the therapy performance assistance server 140 acquires a maximum executable load of “99.4”.

The therapy performance assistance server 140 refers to therapy based on the health-related information of the user C and the maximum executable load calculated, and extracts, as therapy that can be proposed to the user, therapy α using an application A, therapy β and θ using an application B, therapy ε and φ using the application D, and therapy y that are therapeutic applications or therapy with respect to smoking and hypertension. The therapy performance assistance server 140 transmits information indicating the maximum executable load of the user C to a therapeutic application server 150 of the therapeutic application D that is already in use.

The therapeutic application server 150 determines that a load of the therapy ε having a load larger than a load of the therapy φ performed by using the application D is equal to or less than the maximum executable load, and the therapeutic application server 150 determines that the therapy φ is changed to the therapy c having the highest load of loads being within the maximum executable load, and transmits information instructing the change of the therapy, to a therapeutic application function unit 327 in the user terminal 120. The therapeutic application function unit 327 changes the therapy to be executed, according to the instruction to change the therapy.

According to the present embodiment, the therapy performed by the therapeutic application already used by the user can be changed to appropriate therapy, depending on a condition of the user, and the user can be assisted in performing the therapy. In a case where a life event causing a positive impact level has occurred, treatment having a higher load can be executed, and thus therapy can be changed to therapy having a higher load, and a higher therapeutic effect can be obtained. In a case where a life event causing a negative impact level has occurred, a maximum executable load of the user is highly likely to decrease, and the user is highly likely to be unable to continue therapy currently performed, and thus the therapy is changed to therapy appropriate for the maximum executable load of the user, and accordingly even though a therapeutic effect decreases, the therapy can be continued.

The therapy performance assistance server 140 may transmit the information indicating the maximum executable load to another device provided in a system executing the therapeutic application, instead of the therapeutic application server 150. For example, the therapy performance assistance server 140 may transmit the information indicating the maximum executable load to the user terminal 120 in which the therapeutic application D is installed, and the therapeutic application function unit 327 in the user terminal 120 may determine a change of therapy.

Third Embodiment

The present embodiment differs from the first and second embodiments in that, in a case where a user encounters an event having a negative impact level, proposal of an application is performed based on a past medical history of health-related information of the user stored in a health-related information storage unit 347. Here, it is assumed that as user-related information, information indicating that a user D had a smoking habit as a past medical history is provided, and information indicating that the smoking habit has been cured by performing therapy β using an application B is provided. Additionally, it is assumed that the past medical history includes, in addition to information indicating a previous disease, information indicating whether the disease is a lifestyle-related disease.

The user D registers life event information indicating that a life event of divorce has occurred with a user information management server 130 via a user terminal 120. In response to receiving and acquiring the life event information from a personnel information system, a therapy performance assistance server 140 acquires an impact level of “−70” of the life event of “Divorce” from an impact level storage unit 345. The therapy performance assistance server 140 transmits preparation information to the user terminal 120 of the user D, and the user D inputs and transmits a response with respect to description of the preparation information via the user terminal 120.

When the therapy performance assistance server 140 receives response information of the user D, the therapy performance assistance server 140 acquires an impact level correction value of “1” from a preparation information storage unit 346 based on the response information, and calculates a corrected impact value of “−70”, and subsequently, based on the corrected impact value, the therapy performance assistance server 140 calculates a maximum executable load of the user D of “96.7”. The therapy performance assistance server 140 refers to a therapy table and retrieves therapy having loads of from 1 to 96.7, and the therapy performance assistance server 140 extracts therapy θ (94) using an application B for smoke habit treatment (smoking cessation treatment), therapy δ (93) using an application C for a fatty liver, therapy γ (96) using an application E for hypertension, and therapy η (94) using an application F for hypertension.

Based on the health-related information of the user D, the therapy performance assistance server 140 determines that the user D currently has no disease and that the user D had a smoking habit and that the smoking habit is a lifestyle-related disease. Then, based on a negative value of the impact level caused to the user D by the life event of “Divorce” having occurred to the user D, the therapy performance assistance server 140 determines to select therapy with respect to a smoking habit including information indicating that the smoking habit is a lifestyle-related disease in the past medical history information. Whether the life event having occurred to the user has a negative impact level may be determined based on a standard impact level, or may be determined by using a corrected impact level.

The therapy performance assistance server 140 selects, from the therapy extracted, the therapy θ using the application B and having a maximum load among therapy with respect to smoking habit having a load equal to or less than the maximum executable load, and transmits information for performing presentation of therapy to the user terminal 120 of the user D. Alternatively, information indicating the maximum executable load of the user D may be transmitted to a therapeutic application server of the application B, and based on the information, the therapeutic application server may determine therapy and may transmit information proposing initiation of treatment to the user terminal 120.

When a life event having a negative impact level has occurred, a lifestyle-related disease highly tends to deteriorate. It is considered that even in a case where a lifestyle-related disease was ameliorated or cured by treatment or the like in the past, after occurrence of a life event having a negative impact level. behavior such as a smoking habit that adversely affects health is initiated or the lifestyle-related disease recurs. According to the present embodiment, occurrence of a life event having a negative impact level can be detected, and therapy with respect to a lifestyle-related disease having a high recurrence risk can be executed based on a past medical history. Additionally, based on a maximum executable load estimated to be decreased by a negative impact level, therapy estimated to be executable by the user can be proposed, and thus the user can perform appropriate treatment by executing the therapy proposed. Therapy suitable for the user is proposed by using the present embodiment in this manner, and thus assistance in performing the therapy can be performed.

In the processing or operations described above, the processing or operations can be modified freely as long as there is no occurrence of contradiction in the processing or operations such as utilization at a certain step of data that still cannot be utilized at that step. The function unit of each device can be provided in any device as long as there is no occurrence of contradiction in the processing or operations. Additionally, each example described above is exemplified for describing the present invention, and the present invention is not limited to these examples. The present invention can be performed in various forms without departing from the spirit of the present invention.

### REFERENCE SIGNS LIST

100 System

110 Network

120 User terminal

121 Processor

122 Display device

123 Input device

124 Storage device

125 Communication device

128 Bus

130 User information management server

131 Processor

132 Display device

133 Input device

134 Storage device

135 Communication device

138 Bus

140 Therapy performance assistance server

141 Processor

142 Display device

143 Input device

144 Storage device

145 Communication device

148 Bus

150 Therapeutic application server

160 Medical professional terminal

321 Control unit

322 Input unit

323 Display unit

324 Communication unit

325 Browser function unit

326 Email function unit

327 Therapeutic application function unit

331 Control unit

332 Input unit

333 Display unit

334 Communication unit

335 User information storage unit

341 Control unit

342 Input unit

343 Display unit

344 Communication unit

345 Impact level storage unit

346 Preparation information storage unit

347 Health-related information storage unit

348 Therapy information storage unit

1. A system for assisting in performing therapy using a therapeutic application, based on a life event having occurred to a user, the system comprising:
a health-related information storage unit configured to store health-related information of a user; and
an impact level storage unit configured to store an impact level indicating a magnitude of an impact caused to the user by each of a plurality of life events, wherein
the system is configured to:
acquire life event information indicating the life event having occurred to the user;
acquire an impact level of the life event having occurred to the user, from the impact level storage unit based on the life event information;
determine, based on the impact level, a maximum executable load estimated to be executable by the user; and
determine therapy using a therapeutic application and to be proposed to the user performing, based on the maximum executable load, the health-related information of the user stored in the health-related information storage unit, and a therapeutic load of therapy using a therapeutic application, or provide a system of a therapeutic application used by the user and determined based on the health-related information of the user stored in the health-related information storage unit with the maximum executable load to be used for determining therapy performed by using the therapeutic application.

2. The system according to claim 1, wherein
the system is further configured to:
present, in response to the acquisition of the life event information, cognitive inquiry information for acquiring cognition of the user with respect to the life event, and
acquire response information of the user with respect to the cognitive inquiry information, wherein
the determining a maximum executable load comprises correcting the impact level based on the response information of the user acquired, and determining a maximum executable load based on the impact level corrected.

3. The system according to claim 2, wherein
the cognitive inquiry information comprises cognitive intervention information for correcting the cognition of the user.

4. The system according to claim 2, wherein
the determining a maximum executable load comprises determining a motive value of the user based on the impact level, and determining a maximum executable load based on a skill value indicating skill of the user and the motive value of the user, and
the determining therapy using a therapeutic application comprises determining therapy using a therapeutic application and including a therapeutic load smaller than the maximum executable load determined.

5. The system according to claim 4, wherein
the determining a maximum executable load comprises determining a maximum executable load based on Equation (1), and a behavior modification threshold is a value indicating a boundary between occurrence and nonoccurence of behavior modification, and is a predefined constant:
maximum executable load=skill value−behavior modification threshold/motive value (1).

6. The system according to claim 1, wherein
information proposing the determined therapy using the therapeutic application is presented to the user.

7. A method executed by a system for assisting in performing therapy using a therapeutic application, based on a life event having occurred to a user, the system comprising a health-related information storage unit configured to store health-related information of a user, and an impact level storage unit configured to store an impact level indicating a magnitude of an impact caused to the user by each of a plurality of life events, the method including steps for:
acquiring life event information indicating the life event having occurred to the user;
acquiring an impact level of the life event having occurred to the user, from the impact level storage unit based on the life event information;
determining, based on the impact level, a maximum executable load estimated to be executable by the user; and
determining therapy using a therapeutic application and to be proposed to the user performing, based on the maximum executable load, the health-related information of the user stored in the health-related information storage unit, and a therapeutic load of therapy using a therapeutic application, or providing a system of a therapeutic application used by the user and determined based on the health-related information of the user stored in the health-related information storage unit with the maximum executable load to be used for determining therapy performed by using the therapeutic application.

8. A non-transitory computer readable medium storing a program for causing a processor to execute the method according to claim 7.

9. An apparatus for assisting in performing therapy using a therapeutic application, based on a life event having occurred to a user, the system comprising a health-related information storage unit configured to store health-related information of a user, and an impact level storage unit configured to store an impact level indicating a magnitude of an impact caused to the user by each of a plurality of life events, the apparatus being configured to:
acquire life event information indicating the life event having occurred to the user;
acquire an impact level of the life event having occurred to the user, from the impact level storage unit based on the life event information;
determine, based on the impact level, a maximum executable load estimated to be executable by the user; and
determine therapy using a therapeutic application and to be proposed to the user performing, based on the maximum executable load, the health-related information of the user stored in the health-related information storage unit, and a therapeutic load of therapy using a therapeutic application, or provide a system of a therapeutic application used by the user and determined based on the health-related information of the user stored in the health-related information storage unit with the maximum executable load to be used for determining therapy performed by using the therapeutic application.
